(19) United States
(12) Patent Application Publication
(10) Pub. No.: US 2013/0183715 A1
(43) Pub. Date: Jul. 18, 2013

(54) Method and Apparatus for Producing Engineered Fuel from High Cellulose Feedstock

(71) Applicant: Paul T. Baskis, Vero Beach, FL (US)

(72) Inventor: Paul T. Baskis, Vero Beach, FL (US)

(21) Appl. No.: 13/739,296

(22) Filed: Jan. 11, 2013

Related U.S. Application Data
(60) Provisional application No. 61/585,847, filed on Jan. 12, 2012.

(51) Int. Cl. C12P 19/00

(52) U.S. Cl. CPC C12P 19/00
USPC 435/72; 435/290.4; 435/167

(57) ABSTRACT

An apparatus and method for producing methane gas, synthetic hydrocarbon gas, and fertilizer is provided. The apparatus includes a mix tank for mixing cellulosic material with a solvent into a slurry and a generator having an exhaust. The apparatus further includes a stir tank reactor for converting the slurry to a solution containing lignin-like carbon and liquid, and a separator for separating the lignin-like carbon and liquid. An anaerobic digester decomposes the received liquid received from the stir tank into methane and liquid components. A carbon dioxide scrubber scrubs the methane component of carbon dioxide.

The method includes mixing cellulosic material with a solvent into a slurry, and converting the slurry to a solution containing lignin-like carbon and liquid. It also includes separating the lignin-like carbon and liquid and decomposing the liquid into methane and liquid components, and scrubbing the methane component of carbon dioxide.

### CROSS-REFERENCE TO RELATED APPLICATIONS

The present Application claims the benefit of U.S. Provisional Application No. 61/585,847, filed Jan. 12, 2012, the contents of which are incorporated herein by reference.

### FEDERALLY SPONSORED RESEARCH OR DEVELOPMENT

N/A

### TECHNICAL FIELD

The invention relates generally to a method and apparatus for the production of methane gas, synthetic hydrocarbon gas, synthetic petroleum, fertilizer, and high value solid carbon from cellulosic and organic material.

### BACKGROUND OF THE INVENTION

Fossil fuels are expensive. They are also finite. Alternative, renewable, and relatively inexpensive fuel sources are highly desired.

The present invention uses cellulosic materials to produce a range of fuels including methane gas, synthetic petroleum, and synthetic hydrocarbon gas. It also provides a high value solid carbon that can be used as fertilizer, or further processed into a fuel such as coke.

### SUMMARY OF THE INVENTION

In an embodiment of the present invention, an apparatus for producing methane gas, synthetic hydrocarbon gas, and fertilizer is provided. The apparatus includes a mix tank for mixing cellulosic material with a solvent into a slurry and a generator having an exhaust. The apparatus further includes a stir tank reactor for converting the slurry to a solution containing lignin-like carbon and liquid, and a separator for separating the lignin-like carbon and liquid. An anaerobic digester decomposes the received liquid received from the stir tank into methane and liquid components. A carbon dioxide scrubber scrubs the methane component of carbon dioxide.

In another embodiment of the present invention, a method for producing methane gas, synthetic hydrocarbon gas, and fertilizer is provided. The method includes the steps of mixing cellulosic material with a solvent into a slurry, and converting the slurry to a solution containing lignin-like carbon and liquid. The method also includes the steps of separating the lignin-like carbon and liquid and decomposing the liquid into methane and liquid components. The method further includes the steps of scrubbing the methane component of carbon dioxide.

### BRIEF DESCRIPTION OF THE DRAWINGS AND ATTACHMENTS

To understand the present invention, it will now be described by way of example, with reference to the accompanying drawings in which:

FIG. 1 is a flow chart of the methane gas, synthetic hydrocarbon gas, synthetic petroleum and, high value carbon producing apparatus and process of an embodiment of the present invention.

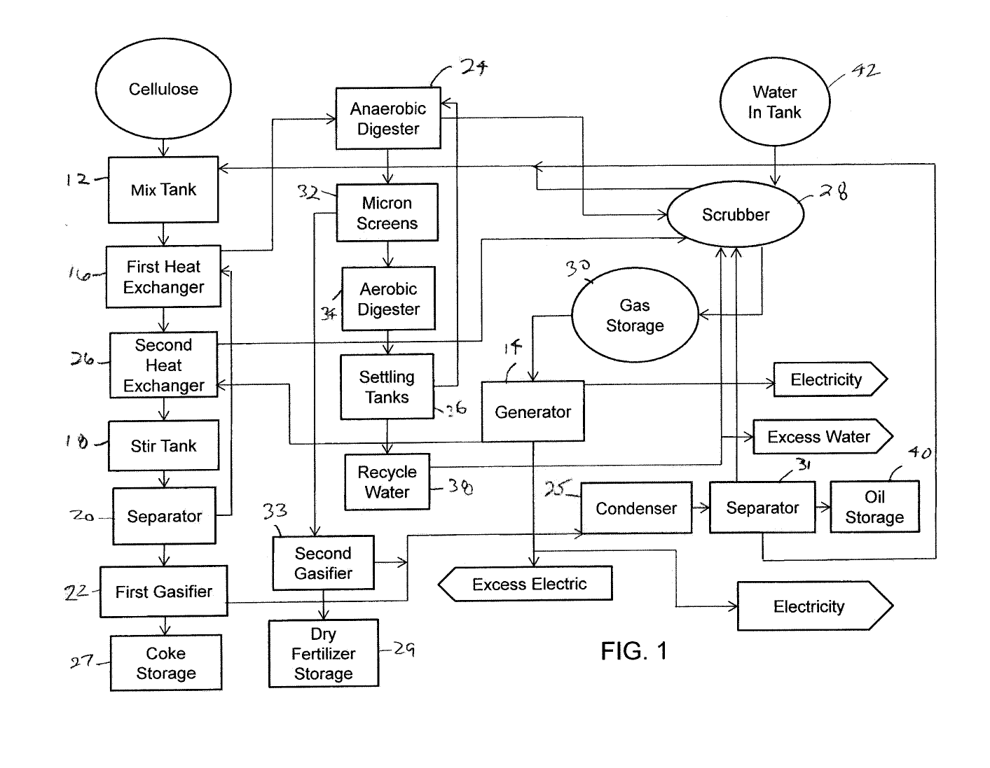

FIG. 2 is a schematic of the liquid treatment system consisting of an anaerobic digester and aerobic digester with associated surge tanks and the recycle water tank.

FIG. 3 is a schematic of the gasifier of an embodiment of the present invention.

FIG. 4 is a schematic of the second gasifier of an embodiment of the present invention.

### DETAILED DESCRIPTION

While this invention is susceptible of embodiments in many different forms, there is shown in the drawings and will herein be described in detail preferred embodiments of the invention with the understanding that the present disclosure is to be considered as an exemplification of the principles of the invention and is not intended to limit the broad aspect of the invention to the embodiments illustrated.

Referring to the FIGS., a process and apparatus 10 for producing methane gas, synthetic hydrocarbon gas, synthetic petroleum, and high value carbon is shown. Cellulosic material in forms that can include wood, wood chips, sawdust, or other suitable cellulosic materials is placed into a mix tank 12. The cellulosic material is blended with carbonic acid to form a slurry, which begins to break down the cellulosic material. The carbonic acid is produced from the exhaust gas of a generator 14 by transferring it through a carbon dioxide scrubber 28 that is under approximately 50 pounds per square inch pressure. This encourages the carbon dioxide to remain in the liquid as carbonic acid. The generator 14 is powered by gas created by the process 10 as will be described below. The generator 14 is produces most of the heat for the process. Heat from the exhaust of generator 14 is used in a first heat exchanger 16 to provide heat to heat up the incoming carbonic acid from the carbon dioxide scrubber 28. The cooled exhaust after passing through the first heat exchanger 16 is then directed to the scrubber 28 where most of the carbon dioxide is removed and is used to help break down the cellulosic material in the mix tank 12.

The slurry of carbonic acid and cellulosic material from the mix tank 12 is transferred though the first heat exchanger 16 to a stir tank reactor 18. The first heat exchanger 16 is heated using the exhaust of the generator 14. The mix tank 12 may also include a heater to achieve and maintain the desired temperature. The slurry is stirred and heated in the stir tank reactor 18. Stirring is done at a rate sufficient to keep the slurry in suspension, and heated to approximately 400 degrees Fahrenheit, and remains in the stir tank reactor 18 for approximately one hour, although dwell times in the stir tank can be varied as desired or required depending on the cellulosic material used or other factors. After remaining in the stir tank 18 for the desired time, the reacted slurry is converted to a lignin-like carbon and a solution of simple sugars and fatty acids and liquid.

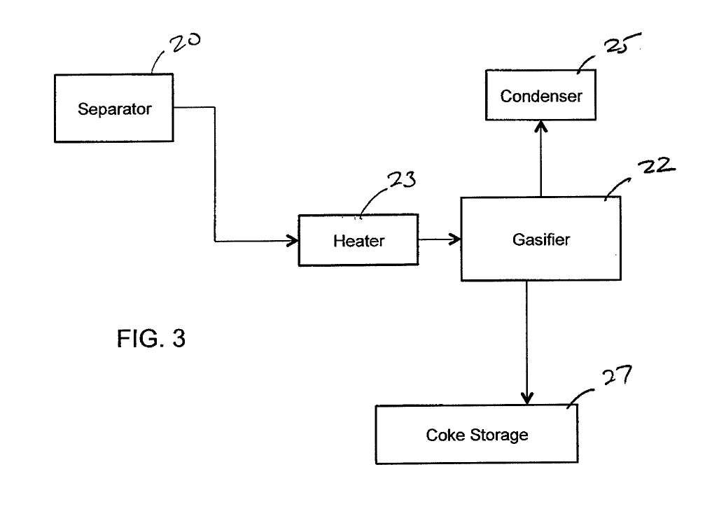

The lignin-like carbon and liquid is transferred from stir tank 18 to a separator 20, where the lignin-like carbon and liquid and a solution of simple sugars and fatty acids are separated. The lignin-like carbon is transferred from the bottom of the separator 20, where most of the liquid was removed, to a gasifier 22. The lignin-like carbon is heated using a heater 23 (FIG. 3) to over 1,000 degrees Fahrenheit to produce a high value coke material of 14,000 to 15,000 BTU per pound. From the heater 23, the coke material is transferred into the gasifier 22 where it is allowed to further degas. The gas vapors are removed through the top of the gasifier 22 and transferred to a condenser 25. The hot gases are cooled so that the liquid components of the gas and the oil are condensed. Generally the higher the gasification temperature the more gas and liquid fuel is produced.

The coke material now with high fixed carbon and low volatile component is transferred out of the gasifier 22 to a coke storage tank 27 via auger. The temperature of the heater 23 and gasifier 22 can also be raised or lowered for the production of carbon products with different volatile components. Generally higher temperature provides for the production of high value coke while lower temperature provides for boiler fuel coke. The volatile component can be tailored to the user's specifications.

The condensed liquid and gas leaving the condenser 25 are transferred to an oil, liquid and gas separator 31. In the separator 31, the oil, liquid and gas are allowed to separate by gravity to form a liquid phase and a gas phase. The liquid phase is comprised of two phases those being (1) a liquid oil that floats on top of the (2) liquid. The liquid is removed from the separator 31 through the bottom of the separator 31, and the oil is removed off of the top of the water by using an overflow well. The gas is removed off of the top of the separator 31 by transferring it the carbon dioxide scrubber 28. The oil is transferred to an oil product storage tank 40 for delivery to a refinery. The liquid removed from the bottom of the separator 31 is transferred to a water in tank 42, where it is reused in the scrubber 28.

From the separator 20, the solution of simple sugars and fatty acids and liquid is transferred to an anaerobic digester 24 after passing through the first heat exchanger 16. The liquid contains the majority of the mineral components of the original cellulosic material. The liquid also contains short chain fatty acids, such as acetic acid, succinic acid, and glutaric acid, and simple sugars. The liquid from the separator 20 gives up its heat to the heat exchanger 16, and emerges from the heat exchanger 16 at a temperature of approximately 105 degrees Fahrenheit. The heat exchanger 16 heats incoming carbonic acid from the carbon dioxide scrubber 28. It also provides temperature control for the incoming solution of simple sugars and fatty acids and mineral and nutrient rich liquid to the anaerobic digester 24.

In the anaerobic digester 24, the solution of simple sugars and fatty acids and liquid undergoes rapid decomposition using methanogenic bacteria creating a biogas having approximately 60% to 65% methane (CH₄) and 35% to 40% carbon dioxide (CO₂). The methane component is directed to the carbon dioxide scrubber 28, where most of the carbon dioxide is removed before the gas is transferred to gas storage 30. The methane component can be used on-site to produce electricity through generator 14, or can be cleaned up for introduction into existing pipelines. The carbon dioxide component is used in the mix tank 12 to digest the incoming cellulose. Typical digestion times are 1 to 2 days, but can be adjusted as desired. Moreover, the digester 24 digests approximately 90% of the material entering it. Tests indicate that approximately 20 cubic feet of methane is produced per pound of digested cellulosic materials.

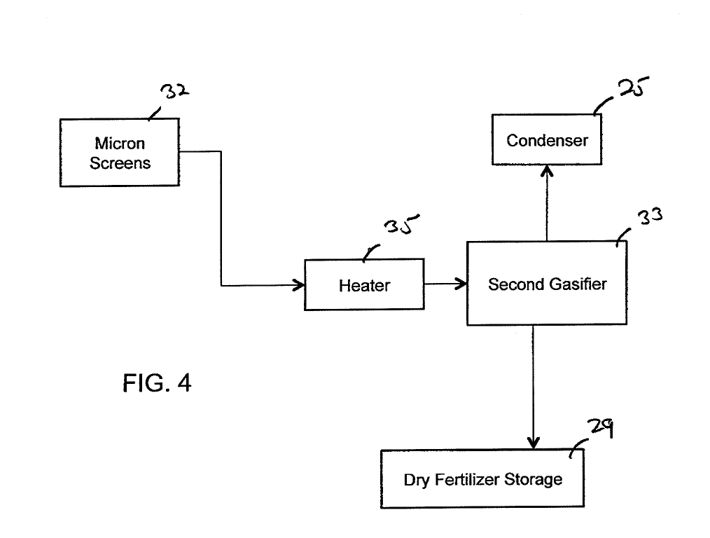

From the digester 24, the liquid component remaining has lost most of its organics in the form of methane. Bacteria that were grown on the nutrients transferred through the anaerobic digester 24 will eventually slough off and leave the anaerobic digester 24 through the bottom. The sloughed off bacteria are transferred through a series of micron screens 32 where the bacteria are separated from the liquid. The bacteria now containing most of the mineral nutrients that were in the liquid are separated at the micron screens 32 are transferred to a second gasifier 33 where they are heated to around 1,000 degrees Fahrenheit by a heater 35 (FIG. 4) to provide for the formation of fertilizer. The fertilizer material leaving the heater 35 is transferred into the second gasifier 33 where it is allowed to degas. The gases leaving the second gasifier 33 are transferred through the condenser 25 where any liquid water and oil are condensed. The fertilizer component leaves the second gasifier 33 through the bottom and is transferred into the dry fertilizer storage tanks 29. Gases from second gasifier 33 are directed to the carbon dioxide scrubber 28.

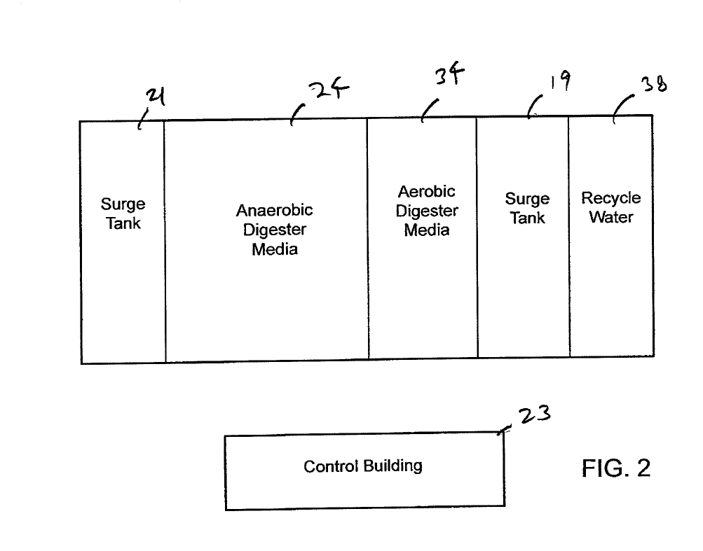

FIG. 2 shows the anaerobic digester and aerobic digester system for treating the liquid from the cellulose. The liquid leaving the separator 20 travels through the first heat exchanger 16 and travels to the surge tank 21 of the anaerobic digester 24. The liquid and fatty acids in the anaerobic digester 24 remain there until the material is digested and biogas is formed. Then the digested material is transferred into the aerobic digester 34 for treatment to lower the BOD and then is transferred into the aerobic digester surge tank 19. Finally the treated liquid is transferred to the recycle water tank 38 where it is used for process liquid or discharged as excess liquid from the process system. A control building 23 contains the controls for the entire process.

After passing through the micron screens 32, the liquid is directed to an aerobic digester 34 for further cleaning. The liquid is essentially free of solids and can be pumped by a standard water pump and plastic ball valves due to lack of abrasives. The output of the aerobic digester 34 is directed to settling tanks 36 where solids are further removed. The solids from the settling tanks 36 are transferred to the anaerobic digester 24 to be converted into additional methane.

The liquid in the settling tanks 36 is transferred to a recycle tank 38 and the carbon dioxide scrubber 28. After being scrubbed in the scrubber 28, the liquid is transferred back to the mix tank 12 to complete the process cycle. Any excess liquid from the settling tanks 36 is discharged, and can be filtered for additional use by any desired or suitable means.

The following chart shows the composition of the solid carbon that results from the process and apparatus of the present invention. This chart shows the raw wood compared with the carbon product resulting from the wood being processed.

| | | | | | | | | | | | As Rec'd | % Dry |
|---|---|---|---|---|---|---|---|---|---|---|---|---|
| | % | As Rec'd | % Dry | As Rec'd | % Dry | As Rec'd | % Dry | MAF | As Rec'd | % Dry | Fixed | Fixed |
| | Moisture | Ash | Ash | Sul | Sul | BTU | BTU | BTU | Vol | Vol | Carb | Carb |
| Raw | 39.12 | 2.46 | 4.04 | — | — | 5,241 | 8,609 | 8,971 | 47.58 | 78.15 | 10.84 | 17.81 |
| Wood | | | | | | | | | | | | |
| Processed | 1.08 | 3.79 | 3.83 | 0.10 | 0.10 | 14,295 | 14,451 | 15,027 | 9.24 | 9.34 | 85.89 | 86.83 |
| Wood | | | | | | | | | | | | |
| Processed | 0.62 | 3.96 | 3.98 | — | — | 14,551 | 14,642 | 15,249 | 18.12 | 18.23 | 77.31 | 77.79 |
| debarked | | | | | | | | | | | | |
| softwood | | | | | | | | | | | | |

This chart shows that the moisture in the wood decreased from approximately 39% to around 1%. The BTU value of the carbon nearly tripled from that of the raw hardwood, and the percent of fixed carbon increased to more than 80%. For debarked softwoods, similar results were obtained. The resulting carbon product is of metallurgical coke quality. It has a very low sulfur content and high fixed carbon content. The process can be varied to obtain a carbon product that meets desired characteristics. For instance, the cellulosic material can be processed at a lower temperature resulting in a boiler fuel with higher volatile composition. The production of this carbon will result in a decrease in the synthetic petroleum and gas products.

The synthetic gas product produced by the process and apparatus of the present invention has a high BTU content that is rich in hydrocarbons. The gas is suitable for use in electric power generation. The gas can also be upgraded and fed into the natural gas pipeline. Low molecular weight hydrocarbons such as propane and butane can either be burned onsite as fuel for electrical generation, or separated and used or stored as separate products. A test of the synthetic gas composition using a batch process reactor open to the atmosphere yielded the gas composition in the chart below. The apparatus and process of the present invention would not be open to the atmosphere. As a result, the carbon monoxide (CO) and nitrogen (N₂) values are much higher than would normally be expected. In the actual process there would be no nitrogen or carbon dioxide.

TOP COMPONENTS

| | | |
|---|---|---|
| 2 | H₂ | 0.40% |
| 12-16 | CH₄ | 24.01% |
| 26-30 | CO + N₂ | 39.71% |
| 32 | O₂ | 4.41% |
| 34 | H₂S | 0.35% |
| 39 | Ar | 1.66% |
| 41-43 | Lo HC | 5.80% |
| 44 | CO₂ | 16.81% |
| | Misc. | 6.85% |

Fertilizer product produced using the process and apparatus of the present invention is handled similar to the carbon product. Liquid exiting the anaerobic digester will have some sloughed off bacteria. The bacteria will be strained or settled out of the liquid and will be sent to a separate gasifier where it is handled similarly to the carbon to form a vitrified char rich in mineral nutrients from the cellulosic material. The fertilizer product will vary in mineral nutrient content depending, for instance, the type of cellulosic material processed.

Methane product produced by the process and apparatus of the present invention will be around 60-65% methane gas and 35-40% carbon dioxide. The gas can be used onsite to produce electricity or cleaned up for introduction into a natural gas pipeline. As indicated above, the digester 24 digests approximately 90% of the material entering it. Tests indicate that approximately 20 cubic feet of methane is produced per pound of digested cellulosic materials.

Expected yields of the products using the method and apparatus of the present invention are shown in the chart below on a per ton basis.

| Product | Percentage | Pounds/Ton | BTU Content |
|---|---|---|---|
| Carbon | 16.0% | 320 | |
| Fatty acid and sugar | 50.0% | 1,000 | 9,600,000 |
| biogas | | | |
| Fertilizer | 6.0% | 120 | |
| Synthetic petroleum | 7.5% | 150 | |
| Synthetic gas | 20.5% | 410 | 5,900,000 |

Synthetic petroleum produced by the apparatus and method of the present invention is expected to have the following properties:

| | |
|---|---|
| API Gravity | 30-50 |
| Viscosity at 40 degrees Celsius | 5.932 (CST) |
| Viscosity at 100 degrees Celsius | 2.270 (CST) |
| Organic chlorides | 14.00 ppm |
| Total sulfur | 80.00 ppm |
| Asphaltene | <0.30 weight percent |

This process may also be used with substances such as cow manure or other materials containing cellulosic and organic materials to give similar results. Manures would produce more fertilizer component and would have a higher mineral nutrient content than cellulosic materials such as wood.

While the specific embodiments have been illustrated and described, numerous modifications come to mind without significantly departing from the spirit of the invention, and the scope of protection is only limited by the scope of the accompanying Claims.

What is claimed is:

1. An apparatus for producing methane gas comprising:
a mix tank for mixing cellulosic material with a solvent into a slurry;
a generator, the generator having an exhaust;
a stir tank reactor for converting the slurry to a solution containing lignin-like carbon and liquid;
a separator for separating the lignin-like carbon and liquid;
an anaerobic digester for decomposing liquid received from the stir tank into methane and liquid components; and
a carbon dioxide scrubber for scrubbing the methane component of carbon dioxide.

2. The apparatus of claim 1 further comprising at least one screen for screening solids from the liquid component received from the anaerobic digester, and an aerobic digester for treating the liquid component received from the at least one screen to create additional methane components.

3. The apparatus of claim 1 further comprising a gasifier for converting the lignin-like carbon to a coke product.

4. The apparatus of claim 1 wherein the solvent includes carbonic acid.

5. The apparatus of claim 2 wherein the solids from the at least one screen are directed to a second gasifier for converting the solids to a fertilizer product.

6. The apparatus of claim 2 further comprising at least one settling tank for receiving liquid from the aerobic digester to remove any additional solids.

7. The apparatus of claim 6 further comprising a recycle tank to receive liquid from the at least one settling tank.

8. The apparatus of claim 3, wherein the gasifier creates a hydrocarbon gas.

9. The apparatus of claim 1 further comprising:
a condenser for condensing gases received from the gasifier into liquid; and
an oil, water and gas separator for separating gases and the condensed liquid, the condensed liquid separated into oil and water components.

10. The apparatus of claim 1 further comprising a first heat exchanger heated by the generator exhaust to heat the solvent.

11. A method of producing methane gas from cellulosic material comprising the steps of:
mixing cellulosic material with a solvent into a slurry;
converting the slurry to a solution containing lignin-like carbon and liquid;
separating the lignin-like carbon and liquid;
decomposing the liquid into methane and liquid components; and
scrubbing the methane component of carbon dioxide.

12. The method of claim 11 further comprising the step of heating the slurry in the stir tank reactor.

13. The method of claim 11 further comprising the step of screening solids from the liquid component received from the anaerobic digester, and treating the liquid component received from the at least one screen to create additional methane components.

14. The method of claim 11 wherein the solvent includes carbonic acid.

15. The method of claim 11 wherein the screened solids are directed to a gasifier.

16. The method of claim 11 further comprising the step of digesting the liquid component from the screening in aerobic digester to create additional methane components.

17. The method of claim 11 further comprising the step of converting the lignin-like carbon to a coke product.

18. The method of claim 11 further comprising the step of converting the screened solids to a fertilizer product.

19. The method of claim 15, wherein the gasifier creates a hydrocarbon gas.

20. The method of claim 11 further comprising the steps of condensing gases received from the gasifier into liquid, and separating gases and the condensed liquid, and separating the condensed liquid into oil and liquid components.
